(19) United States
(12) Patent Application Publication
(10) Pub. No.: US 2006/0285633 A1
(43) Pub. Date: Dec. 21, 2006

(54) Multiple source CT scanner

(76) Inventors: Predrag Sukovic, Birmingham, MI (US); Neal Clinthorne, Ann Arbor, MI (US); Joseph Webster Stayman, Ann Arbor, MI (US); James A. Bertolina, Portage, MI (US); Miodrag Rakic, Redondo Beach, CA (US); William C. Van Kampen, Saline, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/451,134

(22) Filed: Jun. 12, 2006

Related U.S. Application Data
(60) Provisional application No. 60/689,225, filed on Jun. 10, 2005.

(51) Int. Cl. A61B 6/00; G01N 23/00; G21K 1/12; H05G 1/60

(52) U.S. Cl. 378/9

(57) ABSTRACT

A CT scanner includes a plurality of cone-beam x-ray sources offset along a CT axis. A detector is positioned opposite the x-ray sources. The x-ray sources and detector are rotatable about the CT axis. The x-ray sources direct x-rays through the patient that are received by the detector at a plurality of rotational positions, thereby generating projections from the plurality of x-ray sources that are used to construct the three-dimensional CT image of the patient.

This application claims priority to U.S. Provisional Application Ser. No. 60/689,225 filed Jun. 10, 2005.

### BACKGROUND OF THE INVENTION

The present invention relates generally to CT scanners, and more particularly to a compact CT scanner with more than one source.

Generally, CT scanners are expensive, large and difficult to operate. In order to be large enough to scan the entire body, they also occupy an entire large room. As a result, doctors, such as ENT doctors, dentists or oral surgeons have had to send patients to other facilities for CT scans of their sinuses, jaw bones, teeth or other areas. This increases the cost of the procedures and increases the time, since the CT scan must be scheduled in advance, then the results are sent back to the doctor and then a follow up visit is required.

The Assignee of the present invention has provided compact CT scanners that are small, inexpensive and easy to operate. Thus, ENT doctors, dentists and oral surgeons can operate a compact CT scanner in their office and obtain immediate results. Generally, the compact CT scanner includes a source and detector mounted on opposite arms of a rotating gantry. The gantry rotates the source and detector about the patient's head to obtain a CT scan. The source is a cone beam source and the detector is a flat panel detector having a converter for converting x-ray radiation into visible light.

CT scanners are also used in image guided surgical navigation systems. Generally, a patient obtains a pre-operative CT scan, which is then used in subsequent image guided surgical navigation. Again, because the CT scanner is large and in a different area of the hospital there is a delay between the CT scan and the actual surgery.

### SUMMARY OF THE INVENTION

A CT scanner includes a plurality of cone-beam x-ray sources offset along a CT axis. A detector is positioned opposite the x-ray sources. The x-ray sources and detector are rotatable about the CT axis. The x-ray sources direct x-rays through the patient that are received by the detector at a plurality of rotational positions, thereby generating projections from the plurality of x-ray sources that are used to construct the three-dimensional CT image of the patient.

The use of a plurality of x-ray sources increases the field of view of the CT scanner, while reducing its overall dimensions. Cone-beam artifacts are also reduced.

### BRIEF DESCRIPTION OF THE DRAWINGS

Other advantages of the present invention can be understood by reference to the following detailed description when considered in connection with the accompanying drawings wherein:

FIG. 1 illustrates a side view of the CT scanner of the present invention.

FIG. 2A illustrates an end view of CT scanner of FIG. 1, showing one alignment of the sources, X-axis and detector.

FIG. 2B illustrates an alternate arrangement of the sources and detector.

FIG. 2C is a side perspective view illustrating one possible alignment of the x-ray beams on the detector.

FIG. 2D is a side perspective view similar to FIG. 2C illustrating an alternative alignment of the x-ray beams on the detector.

FIG. 3 illustrates a second embodiment of the CT scanner of the present invention.

### DETAILED DESCRIPTION OF THE PREFERRED EMBODIMENTS

The present invention provides a more compact CT scanner with an increased field of view and with reduced cone beam artifacts in the CT image.

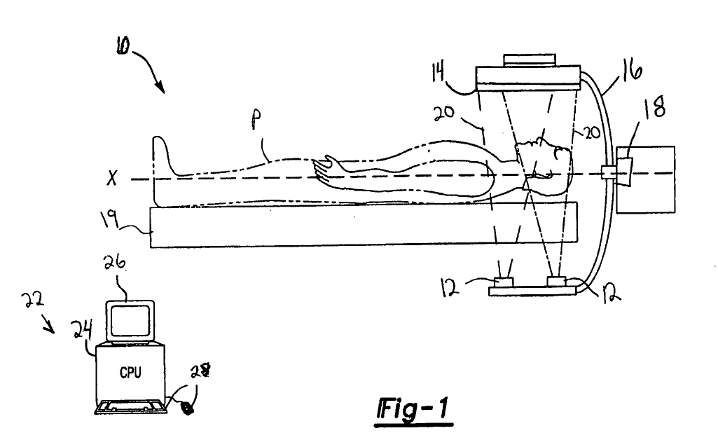

FIG. 1 illustrates a first embodiment of the CT scanner 10 according to the present invention. The CT scanner 10 includes a plurality of x-ray sources 12. Although two are shown for simplicity, more than two x-ray sources 12 could also be utilized according to the teachings herein. The x-ray sources 12 are mounted opposite a detector 14 on a gantry 16. The detector 14 may be a flat panel detector having converter for converting x-ray radiation into visible light. The gantry 16 is mounted to a motor 18 to rotate about an axis X.

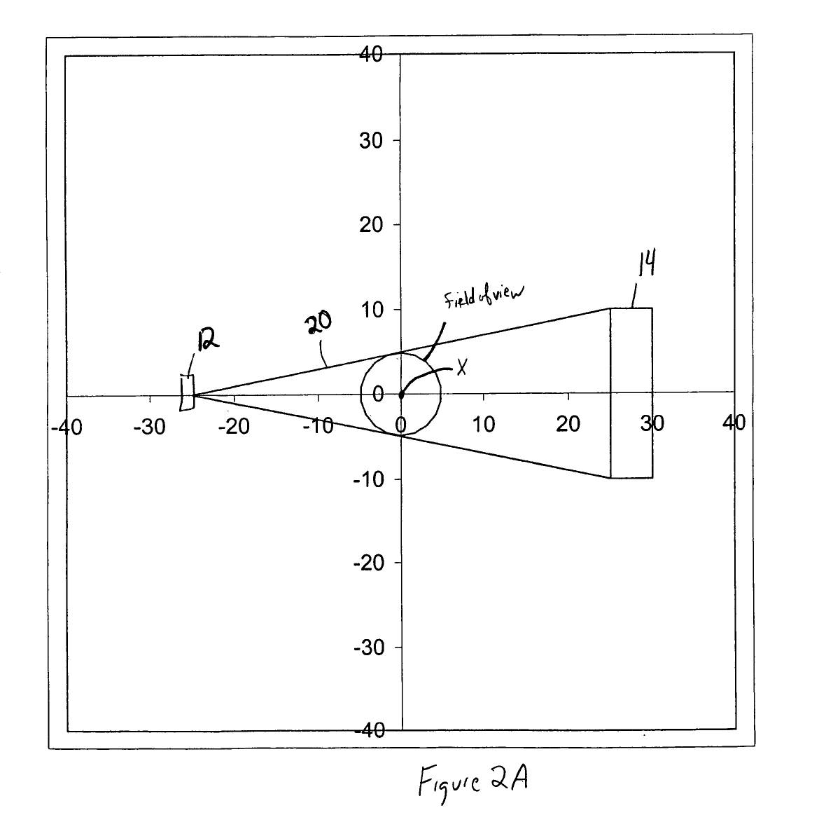

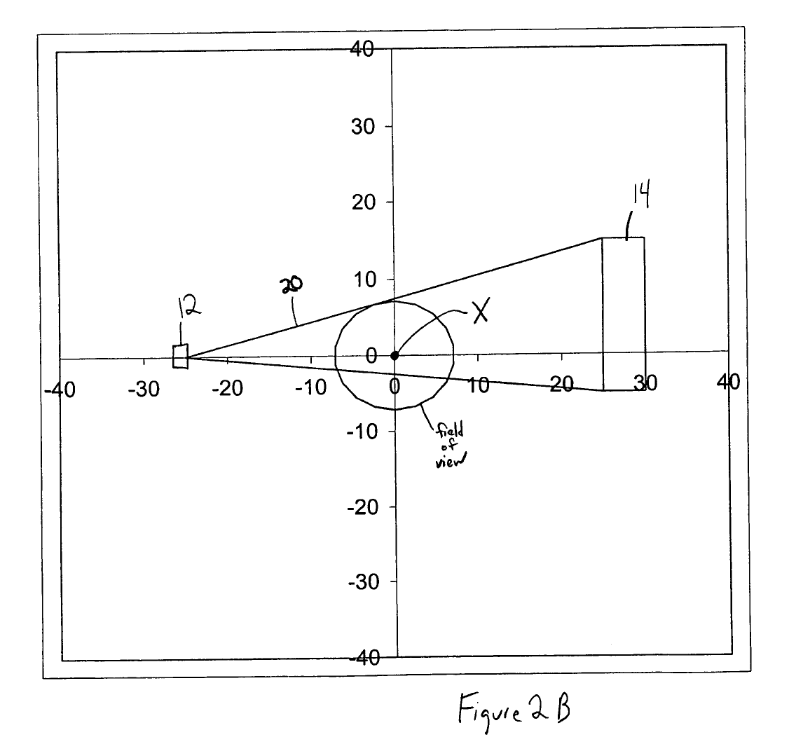

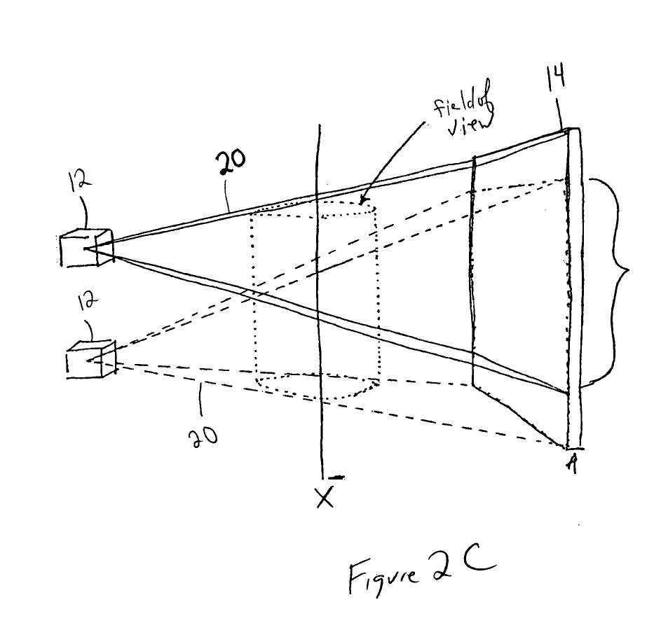

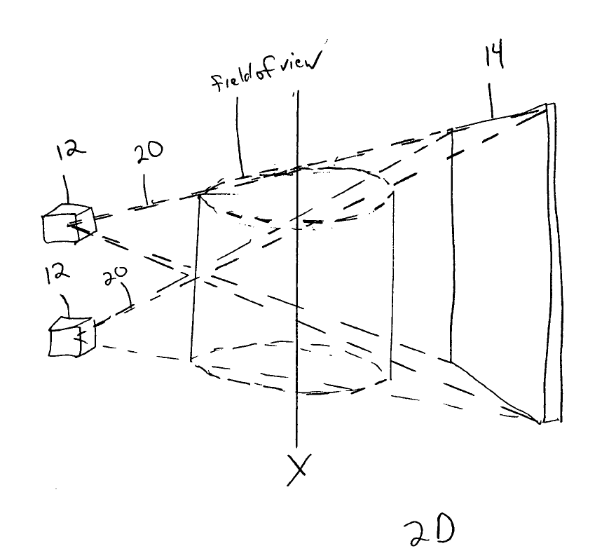

The x-ray sources 12 are axially spaced from one another and may be angularly aligned relative to the axis X. Optionally, the x-ray sources 12 may be angularly offset (and need not be the same radial distance from the axis X). The detector 14 may be mounted diametrically opposite the x-ray sources 12 as shown in FIG. 2A, or the detector 14 could be offset in a direction perpendicular to the axis X, such that the detector 14 is centered off-axis (for example, as shown in FIG. 2B), in order to increase the radius of the cylindrical imaging volume or field of view. Alternatively to the arrangements shown in FIGS. 2A and 2B, the x-ray sources 12 may be spaced slightly farther from the X axis than is the detector 14.

The x-ray sources 12 are arranged such that their x-ray beams 20 have at least significant overlap on the detector 14 in the X direction, as is shown in FIG. 2C. As is demonstrated by FIG. 2C, the x dimension of the field of view (in the direction along the X-axis) is increased compared to a CT scanner with a single source. The y dimension of the field of view (the radius of the cylindrical field of view) is determined by the point of intersection between the two x-ray beams 20. The radius of the cylindrical field of view begins where the x-ray beams 20 intersect.

Alternatively, with 100% overlap between the x-ray beams 20, as shown in FIG. 2D, the radius of the cylindrical field of view can be increased even further. The amount of overlap can be determined and adjusted based upon the imaging volume needed balanced with a desire minimize x-ray dosage.

The CT scanner 10 further includes a computer 22, including a CPU 24 having a processor and memory and/or other storage of a computer program to perform the functions described herein. The CPU 24 further includes a display 26 and input devices 28. The CPU 24 controls the x-ray sources 12 and motor 18 and receives the images from the detector 14.

In use, with a patient P lying on table 19, the CPU 24 commands the motor 18 to begin rotation of the gantry 16. During the rotation, the CPU 24 commands the x-ray sources 12 to activate alternately, sending x-rays through the patient P that are received by the detector 14. The x-ray sources 12 can both be activated alternately at each of a plurality of rotational positions of the gantry 16, or the x-ray sources 12 can alternate in such a manner that each one is activated at alternating rotational positions. Each image from the detector 14 is recorded by the CPU 24 as is some identification of which source 12 was activated and the rotational position of the gantry 16 at the time the image was taken. Alternatively, the gantry 16 can perform multiple revolutions, with one x-ray source 12 performing a CT scan during one revolution and the other x-ray source 12 performing a CT scan during the other revolution. If more than two x-ray sources 12 are used, the x-ray sources 12 would still be alternated during a single revolution or in multiple revolutions. Note that although the beams 20 from the multiple x-ray sources 12 overlap on the detector 14, they will not be activated at the same time. However, if more than two x-ray sources 12 are utilized, then subsets of non-overlapping x-ray sources 12 could be activated simultaneously.

The plurality of images taken by detector 14 by the multiple x-ray sources at a plurality of rotational angles of the gantry 16 are stored in CPU 24. The CPU 24 then generates a 3-D CT image based upon the stored images from the detector 14. The CPU 24 combines the images from the detector 14 that result from the multiple x-ray sources 12, based upon the known relative positions of the multiple x-ray sources 12.

The use of multiple x-ray sources 12 increases the field of view of the CT scanner 10 along the axis X and increases the radius of the imaging volume. Further, the overlap between the beams 20 from the multiple x-ray sources 12 eliminates much of the cone beam artifacts that may otherwise be present at the outer periphery of the cone beams 20. Additionally, the use of multiple x-ray sources 12 permits the location of the x-ray sources 12 closer to the axis X of rotation of the CT scanner 10 (possibly at some trade-off with the size of the field of view).

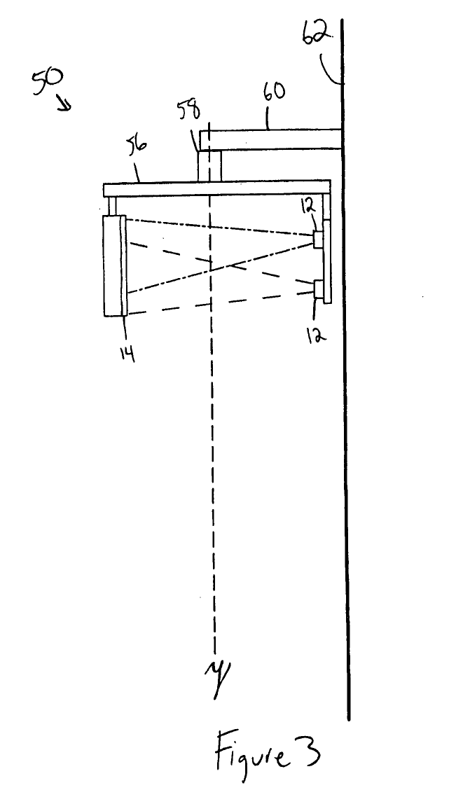

An alternate embodiment of the CT scanner 50 is shown in FIG. 3. In this embodiment, the multiple sources 12 and the detector 14 are mounted on a gantry 56 rotatable about a vertical axis Y, such as for scanning a head of a patient in a sitting or standing position. The gantry 56 is rotatable by a motor 58 mounted on an arm 60 secured to a wall 62. The arm 60 may also be supported by a frame rested on the floor. Because the multiple x-ray sources 12 permit the x-ray sources 12 to be moved closer to the axis X, the arm 60 can also be shortened, thus, reducing the overall size, weight and cost of the CT scanner 50 compared to previous compact CT scanners. All of the arrangements shown and described with respect to FIGS. 2A-D could be utilized in the embodiment of FIG. 3.

For all of the embodiments shown above, tomographic reconstruction of projection from this CT Scanner 10, 50 will need to account for the multi-source geometry. There are several possibilities for multi-source reconstruction:

1) Traditional reconstruction and merging. Because the projections from each of the sources 12 forms a traditional cone-beam dataset, projections provided by each source 12 can be individually reconstructed using traditional methods (e.g.: Feldkamp reconstruction or other direct methods) and merged (using weighted averaging, for example, for the volume covered by more than one source 12).

2) Model-based iterative approaches. If one combines a data model that accounts for each source 12 into a single complete forward model, the entire imaging volume can be reconstructed by optimizing a data consistency objective. Examples of this type of reconstruction include ART (Arithmetic Reconstruction Technique) and it variations, and all of the iterative statistical approaches (like those based on Poisson or Gaussian likelihood functions).

3) Rebinning techniques. One may also take what amounts to multiple cone-beam circular orbits and rebin that data into a more convenient form for reconstruction. This kind of processing has been performed for positron emission tomography (PET) scanning and may be applied here as well. For example, in regions where the cone-beams overlap, data can be re-binned into more convenient projection planes and reconstruction can be comprised of many two-dimensional (slice-by-slice) reconstructions.

Although the x-ray sources 12 in both embodiments are shown as completely separate, independent x-ray sources 12, a single, multiple-source unit could also be utilized. For example, as used herein the term “multiple source” includes units where a power supply or other circuitry are shared between the “multiple sources,” or where one of the electrodes in the x-ray sources is shared, as long as the sources of the x-rays (i.e., the locations from which x-rays are emitted) are spaced apart and can be activated independently. More than one detector 14 could be used, with one detector aligned with each of the x-ray sources 12.

In accordance with the provisions of the patent statutes and jurisprudence, exemplary configurations described above are considered to represent a preferred embodiment of the invention. However, it should be noted that the invention can be practiced otherwise than as specifically illustrated and described without departing from its spirit or scope. Alphanumeric identifiers for steps in method claims are for ease of reference in dependent claims and do not signify a required sequence unless otherwise stated.

What is claimed is:

1. A CT scanner comprising:
a detector adjacent a CT axis;
a first x-ray source rotatable about the CT axis, the first x-ray source aligned with the detector, such that x-rays from the first x-ray source are received by the detector; and
a second x-ray source axially spaced from the first x-ray source, the second x-ray source aligned with the detector such that x-rays from the second x-ray source are received by the detector.

2. The CT scanner of claim 1 further including a controller programmed to activate the first x-ray source and the second x-ray source alternately between the first x-ray source and the second x-ray source.

3. The CT scanner of claim 1 wherein the detector is rotatable about the CT axis.

4. The CT scanner of claim 1 wherein the second x-ray source is rotatable about the CT axis.

5. The CT scanner of claim 4 wherein the first x-ray source and the second x-ray source are cone-beam x-ray sources.

6. The CT scanner of claim 5 wherein the first x-ray source and the second x-ray source are arranged such that cone-beam x-rays from the first x-ray source and the second x-ray source at least partially overlap on the detector.

7. A CT scanner comprising:
at least one detector;
a first cone-beam x-ray source, the first x-ray source aligned with the at least one detector, such that x-rays from the first x-ray source are received by the at least one detector; and
a second cone-beam x-ray source spaced from the first x-ray source, the second x-ray source aligned with the at least one detector such that x-rays from the second x-ray source are received by the at least one detector, wherein the first x-ray source, the second x-ray source and the at least one detector are rotatable about an x-axis.

8. The CT scanner of claim 7 further including a controller programmed to alternatingly activate the first x-ray source and the second x-ray source.

9. The CT scanner of claim 8 wherein cone-beam x-rays from the first x-ray source and the second x-ray source are directed at areas that at least partially overlap on the at least one detector.

10. A method for generating a CT image including the steps of:
a) directing first cone-beam x-rays at a patient from each of a plurality of first locations angularly spaced about an x-axis; and
b) directing second cone-beam x-rays at the patient from each of a plurality of angularly spaced second locations;
wherein the second cone-beam x-rays are offset along the x-axis relative to the first cone-beam x-rays.

11. The method of claim 10 further including the step of repeatedly alternating said steps a) and b).

12. The method of claim 10 further including the steps of:
c) receiving said first cone-beam x-rays and said second cone-beam x-rays after passing through the patient; and
d) generating a CT image based upon said step c).

13. The method of claim 12 wherein said step d) further includes the step of generating a three-dimensional model of the patient based upon the received first cone-beam x-rays and the received second cone-beam x-rays.

14. The method of claim 12 wherein said step c) includes the steps of receiving the first cone-beam x-rays in a first area and receiving the second cone-beam x-rays in a second area, the first area at least partially overlapping with the second area.

15. The method of claim 10 wherein said step a) includes generating said first cone-beam x-rays from a first source and wherein said step b) includes generating said second cone-beam x-rays from a second source, the first source axially offset from the second source.

16. The method of claim 15 further including the step of receiving the first cone-beam x-rays and the second cone-beam x-rays in a plane opposed to the first source and the second source.
